(19) United States
(12) Patent Application Publication
(10) Pub. No.: US 2022/0152000 A1
(43) Pub. Date: May 19, 2022

(54) PROLONGED RELEASE FORMULATION COMPRISING TACROLIMUS

(71) Applicant: PHARMATHEN S.A., Pallini Attikis (GR)

(72) Inventors: Evangelos KARAVAS, Pallini Attikis (GR); Efthymios KOUTRIS, Pallini Attikis (GR); Vasiliki SAMARA, Pallini Attikis (GR); Ioanna KOUTRI, Pallini Attikis (GR); Anastasia KALASKANI, Pallini Attikis (GR); Manolis FOUSTERIS, Pallini Attikis (GR); Maria TSALTA, Pallini Attikis (GR)

(73) Assignee: PHARMATHEN S.A., Pallini Attikis (GR)

(21) Appl. No.: 17/441,033

(22) PCT Filed: Mar. 19, 2020
(86) PCT No.: PCT/EP2020/025137

§ 371 Date: Sep. 20, 2021

(30) Foreign Application Priority Data
Mar. 20, 2019 (GR) 20190100139

(51) Int. Cl. A61K 31/436; A61K 9/00; A61K 9/48

(52) U.S. Cl. CPC A61K 31/436; A61K 9/0053; A61K 9/4866; A61K 9/4858; A61K 9/4833

(57) ABSTRACT

The present invention relates to a method of manufacturing of a prolonged release solid dispersion formulation comprising Tacrolimus or a pharmaceutically acceptable salt thereof. Furthermore it relates to the manufacturing process of such a dosage form.

### TECHNICAL FIELD OF INVENTION

The present invention relates to a prolonged release formulation comprising Tacrolimus or a pharmaceutically acceptable salt thereof. Furthermore, it relates to the manufacturing process of such a dosage form.

### BACKGROUND OF THE INVENTION

Tacrolimus is a macrolide and has been shown to have immunosuppressive and antimicrobial activity. It is of great importance for the prevention of rejection of organ or tissue transplants and is generally used for treatment of graft versus host diseases, autoimmune diseases and infectious diseases. Tacrolimus has been shown to increase survival of the host and transplanted graft in animal transplant models of liver, kidney, heart, bone marrow, small bowel, lung, trachea, skin cornea and limb.

Tacrolimus inhibits T-lymphocyte activation, although the exact mechanism is unknown. Experimental evidence suggests that it binds to an intracellular protein, FKBP-12. A complex of tacrolimus-FKBP-12, calcium, calmodulin and calcineurin is then formed and the phosphatase activity of calcineurin is inhibited. This effect may prevent the dephosphorylation and translocation of nuclear factor of activated T-cells, a nuclear component thought to initiate gene transcription for the formation of lymphokines. The end result is the inhibition of T-lymphocyte activation and subsequent immunosuppression.

Tacrolimus is also known as FK-506 or FR-900506 and it forms white crystals or crystalline powder. A preparation method has been described in patent EP-B-0184162. Tacrolimus is soluble in methanol, ethanol (25 mg/ml), acetone, ethyl acetate, diethyl ether, chloroform, dicloromethane. It is sparingly soluble in hexane, petroleum ether and it is insoluble in water.

It has been observed that absorption of tacrolimus after oral administration is negatively influenced by simultaneous ingestion of food. Thus the rate and extent of tacrolimus absorption is greatly improved under fasted conditions. This together with the fact that it is insoluble in water has made the development of a formulation with an optimum dissolution rate and high bioavailability difficult to develop.

Tacrolimus is currently marketed as soft gelatin capsules comprising anhydrous tacrolimus and marketed as Prograf® 0.5 mg, 1 mg, 5 mg; granules for suspension marketed as Modigraf® 0.2 mg, 1 mg and prolonged release soft capsules marketed as Advagraf® 0.5 mg, 1 mg, 3 mg, 5 mg.

Various patents and patent applications have attempted to address the issue of increasing bioavailability of tacrolimus. European patent EP-B-1064942 discloses a sustained release formulation of tacrolimus obtained by dissolving tacrolimus in molten glycerol monosterate or tetraglycerin trifatty acid ester.

WO-A-01/37808 discloses a formulation obtained by spraying a solution of tacrolimus, Solulan C-24, monoglycerides and deoxycholic acid in organic solvent on non-pareil seeds.

WO-A-03/004001 discloses a controlled agglomeration method for improving the bioavailability of poorly water soluble compounds in solid solution or dispersions.

Oral bioavailability of a drug depends on its solubility and its dissolution rate. Improvement in dissolution rate of poorly soluble drugs after oral administration is one of the most crucial challenges in modern pharmaceutics. Many methods are available to improve these characteristics including salt formation, micronization and addition of solvent or surface-active agents. Also, solid dispersion has been shown to successfully improve dissolution rate and bioavailability of water insoluble drugs. Usually, the term solid dispersion refers to a group of solid products consisting of at least two different components, generally a hydrophilic matrix and a hydrophobic drug. The matrix can be either crystalline or amorphous and drug can be dispersed molecularly, in amorphous particles or in crystalline particles.

There is still the need to provide an alternative formulation of tacrolimus having optimized dissolution drug release profile and addresses the issue of poor bioavailability of the specific active pharmaceutical ingredient. The present invention relates to a prolonged release formulation that comprises a solid dispersion of Tacrolimus that increases its limited water solubility thus enhancing its bioavailability.

### SUMMARY OF THE INVENTION

It is, therefore, an object of the present invention to provide a pharmaceutical formulation comprising tacrolimus or a pharmaceutically acceptable salt thereof that has adequate bioavailability and good physicochemical properties.

A further object of the present invention is to provide a prolonged release pharmaceutical formulation comprising a solid dispersion of tacrolimus or a pharmaceutically acceptable salt thereof. The prolonged release formulation further comprises a polymer or a combination of polymers.

The main objective of the present invention is to provide an oral dosage form comprising tacrolimus or a pharmaceutically acceptable salt thereof and a manufacturing process for the preparation of such formulation. The manufacturing process comprising granulation in an organic solvent of a percentage of the total amount of tacrolimus or pharmaceutically acceptable salt thereof with a hydrophilic polymer, a hydrophobic polymer and an acceptable filler to form a solid dispersion. Surprisingly, it was found that when only 20 to 50% of the total Tacrolimus was dissolved in a solid dispersion and the remaining amount of Tacrolimus is in a non-dispersed, crystalline form the product showed the required solubility, dissolution profile and bioavailability.

Other objects and advantages of the present invention will become apparent to those skilled in the art in view of the following detailed description.

### DETAILED DESCRIPTION OF THE INVENTION

The term “solid dispersion” as used in the present invention denotes a drug or active ingredient or substance dispersed on a particulate level in an inert vehicle, carrier, diluent or matrix in the solid state, i.e. usually a fine particulate dispersion.

Also the term “solid solution” as used in the present invention denotes a drug or active ingredient or substance dissolved on a molecular level in an inert vehicle, carrier, diluent or matrix in the solid state.

As used herein, the term “bioavailability” denotes the degree means to which a drug or other substance becomes available to the target tissue after administration. As used herein, the term “‘bioequivalency” denotes a scientific basis on which generic and brand name drugs are compared with one another.

In the present context, the terms “prolonged release”, “controlled release” and “modified release” are intended to be equivalent terms covering any type of release of tacrolimus from a composition of the invention that is appropriate to obtain a specific therapeutic or prophylactic response after administration to a subject. A person skilled in the art knows how prolonged release/controlled release/modified release differs from the release of plain tablets or capsules. The terms “release in a prolonged manner”, “release in a controlled manner” or “release in a modified manner” have the same meaning as stated above.

The pharmaceutically active ingredient in the present invention is tacrolimus (aka FK-506 or FR-900506). However, within the scope of the present invention is Tacrolimus in any physical form (crystals, amorphous powder, any possible polymorphs, any possible solvates including the hydrate, anhydrate, complexes thereof etc.). Included is also any derivative or active metabolite of Tacrolimus, pharmaceutically acceptable salts, solvates, complexes and prodrugs thereof.

Tacrolimus is extensively metabolized by the CYP3A4 isoenzyme in the gut wall and liver. Accordingly, a suitable controlled release composition may be a composition that is designed to release tacrolimus in a delayed manner so as to avoid or reduce the CYP3A4 metabolism in the gastrointestinal tract. It is believed that such a release profile significantly enhances the bioavailability of tacrolimus in mammals, since all or a major part of the active ingredient is in fact released in the gastrointestinal tract in such a manner that CYP3A4 metabolism is substantially avoided or at least significantly reduced.

The desired release profile of the pharmaceutical composition may be provided by using a pharmaceutical composition comprising a solid dispersion or solid solution of active ingredient, i.e. Tacrolimus or an analogue thereof, in a hydrophilic or water-miscible vehicle and one or more modifying release agents.

A pharmaceutical composition according to the invention releases Tacrolimus in a controlled manner in order to extend the therapeutic action of tacrolimus. In one aspect the release may be pH dependent, i.e. the release predominantly takes place after passage of the stomach. Such a pH dependent release is mainly provided by means of enteric coating material as described herein. The release may also be pH independent, e.g. by providing the composition with a controlled release coating such as, e.g. a cellulose based coating like e.g. ethylcellulose or by providing the composition in the form of a matrix composition such as, e.g., a hydrophilic cellulose polymer matrix type e.g. based on HPMC. A combination may of course also be employed.

It is therefore provided a prolonged release Tacrolimus-containing pharmaceutical composition having the active ingredient dissolved or dispersed in a combination of a hydrophilic and a hydrophobic polymer, which most preferably comprises ethylcellulose and hydroxypropylmethylcellulose.

In particular, one aspect of the invention includes an oral dosage form, wherein the oral dosage comprises a portion of tacrolimus compound dissolved in a solid dispersion form and the remaining amount of the macrolide compound is in a non-dispersed/crystalline form. Both portions of macrolide compound equals 100% of the pharmaceutically effective amount. Apart from the macrolide compound, two modifying release agents are also enclosed in the current pharmaceutical composition as following:

Hydroxypropylmethyl cellulose, a water-miscible, “hydrophilic” polymer which modifies the drug release profile by forming a polymer gel layer in aqueous medium. HMPC 2910 type of having nominal viscosity between 1 to 4,000 cps (2% solution, measured at 20° C. by a viscometer of Brookfield type) is being applied to current formulation to extend the drug release from the solid dispersion system,

Ethyl cellulose, a water-immiscible, “hydrophobic” polymer which modifies the drug release by controlling water penetration in the matrix system and thus causing “erosion” of formulation within the aqueous medium. Ethyl cellulose of low nominal viscosity (when measured as 5% solution at 25° C. in an Ubbelohde viscometer), between 9 to 11 cps (Dow Chemicals/US Trademark: Ethocel std10), is applied to the current formulation to enhance the sustained release effect of tacrolimus compound.

In the present context, the term “hydrophilic” describes that something is familiar to water: a molecule or portion of a molecule is electrically polarized and capable of forming hydrogen bonds with water molecules, enabling it dissolve more readily in water than in oil or other “non-polar” solvents.

In the present context, the term “hydrophobic” denotes a compound tending to be electrically neutral and non-polar, and thus preferring other neutral and nonpolar solvents or molecular environments.

The formulation of the present invention is a hard gelatin capsule filed with particulate material. The particulate material obtained is a free-flowing powder and therefore readily processable into e.g. solid dosage forms such as tablets, capsules or sachets.

Normally, the particulate material has properties that are suitable in order to manufacture tablets by direct compression without addition of large amounts of further additives.

At least a part of tacrolimus is present in the composition in the form of a solid dispersion. Normally, 20% or more, 30% or more, 40% or more, but not more than 50% tacrolimus is present in the composition in the form of a solid dispersion.

A solid dispersion may be obtained in different ways e.g. by employing organic solvents or by dispersing or dissolving the active substance in another suitable medium (e.g. an oil or an oily-like material that is in liquid form at room temperature or at elevated temperatures).

Solid dispersions (solvent method) may for example be prepared by dissolving a physical mixture of the active substance (e.g. a drug substance) and the carrier in a common organic solvent, followed by evaporation of the solvent. The carrier is often a hydrophilic polymer. Suitable organic solvents include pharmaceutical acceptable solvent in which the active substance is soluble such as methanol, ethanol, methylene chloride, chloroform, ethylacetate, acetone or mixtures thereof.

Suitable water soluble carriers include polymers such as polyethylene glycol, poloxamers, polyoxyethylene stearates, poly-ε-caprolactone, polyvinylpyrrolidone (PVP), polyvinylpyrrolidone-polyvinylacetate copolymer PVP-PVA (Kollidon VA64), poly-methacrylic polymers (Eudragit R S, Eudragit R L, Eudragit N E, Eudragit E) and polyvinyl alcohol (PVA), hydroxypropyl cellulose (HPC), hydroxypropyl methyl cellulose (HPMC), methyl cellulose, ethyl cellulose and poly(ethylene oxide) (PEO).

Polymers containing acidic functional groups may be suitable for solid dispersions, which release the active substance in a preferred pH range providing acceptable absorption in the intestines. Such polymers may be one or more selected from the group comprising hydroxypropyl methylcellulose phtalate (HMPCP), polyvinyl acetate phtalate (PVAP), hydroxypropylmethylcellulose acetate succinate (HPMCAS), alginate, carbomer, carboxymethylcellulose, methacrylic acid copolymer (Eudragit L, Eudragit S), shellac, cellulose acetate phthalate (CAP), starch glycolate, polacrylin, methyl cellulose acetate phtalate, hydroxypropyulcellulose acetate phthalate, cellulose acetate terephtahalate, cellulose acetate isophthalate and cellulose acetate trimellitate.

Relative to the amount of the active substance and the polymer in the solid dispersion, the weight ratio of active substance to polymer or combination of polymers may be in a range of from about 3:1 to about 1:20. However, narrower range of from about 3:1 to about 1:15 may also be used. Preferably, the ratio thereof is from 1:10 to 1:20.

Examples of suitable excipients for use in a composition or solid dosage form according to the present invention include fillers, diluents, disintegrants, binders, lubricants and the like and mixtures thereof. As the composition or solid dosage form according to the invention may be used for different purposes, the choice of excipients is normally made taken such different uses into considerations. Other pharmaceutically acceptable excipients for suitable use are e.g. acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents and the like.

Examples of suitable fillers, diluents and/or binders include lactose (e.g. spray-dried lactose, α-lactose, β-lactose, microcrystalline cellulose hydroxypropylcellulose, L-hydroxypropylcellulose (low substituted), hydroxypropyl methylcellulose (HPMC), methylcellulose polymers, hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen.

Specific examples of diluents are e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of disintegrants are e.g. alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch.

Specific examples of binders are e.g. acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch.

Glidants and lubricants may also be included in the composition. Examples include stearic acid, magnesium stearate, calcium stearate or other metallic stearate, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate.

Other excipients which may be included in a composition or solid dosage form of the invention are e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release.

Other additives in a composition or a solid dosage form according to the invention may be antioxidants like e.g. ascorbic acid, ascorbyl palmitate, butylated hydroxyanisole, butylated hydroxytoluene, hypophosphorous acid, monothioglycerol, potassium metabisulfite, propyl gallate, sodium formaldehylde sulfoxylate, sodium metabisulfite, sodium thiosulfate, sulfur dioxide, tocopherol, tocopherol acetate, tocopherol hemisuccinate, TPGS or other tocopherol derivatives. The carrier composition may also contain e.g. stabilizing agents.

The solid dispersion is preferably formed by spray drying techniques, controlled agglomeration, freeze-drying, drying or coating on carrier particles or any other solvent removal process. The dried product contains the active substance present in the form of a solid dispersion including a molecular dispersion and a solid solution.

The pharmaceutical compositions comprising Tacrolimus at least partly in form of a solid dispersion or solution may in principle be prepared using any suitable procedure for preparing pharmaceutical compositions known within the art. Apart from using the organic solvent based method, solid dispersion or solid solutions of Tacrolimus may be obtained by dispersing and/or dissolving tacrolimus in the carrier composition used in the controlled agglomeration method. Stabilizing agents etc. may be added in order to ensure the stability of the solid dispersion/solution.

The pharmaceutical composition according to the invention is in particulate form and may be employed as such. However, in many cases it is more convenient to present the composition in the form of granules, pellets, microspheres, nanoparticles and the like or in the form of solid dosage forms including tablets, capsules and sachets and the like.

Normally, a pharmaceutical composition or a solid dosage form of the invention is intended for administration via the oral, buccal or sublingual administration route.

Particle sizes of substances can be measured using various commonly available methods such as measurement using light (eg. light-scattering methods or turbidimetric methods), sedimentation methods (eg. pipette analysis using an Andreassen pipette, sedimentation scales, photosedimentometers or sedimentation in a centrifugal force), pulse methods (eg. Coulter counter), or sorting by means of gravitational or centrifugal force. There are various known methods for the control of the particle size of substances including reduction by comminution or de-agglomeration by milling and/or sieving, or particle size increase by agglomeration through granulation, blending or a mixture thereof.

The process for preparing a solid dispersion comprising Tacrolimus or a pharmaceutically acceptable salt thereof, and a combination of a hydrophilic polymer a hydrophobic polymer and a filler, the process comprising:

a) dissolving from about 20% to about 50% of the total amount of Tacrolimus compound to be used in the formulation in an organic solvent (anhydrous ethanol) to form a macrolide solution,

b) pre-mixing the polymers, with at least 50% of the filler stated amount,

c) mixing the Tacrolimus solution of step (a) with the premixed blend of solid materials of step (b) to form a solid dispersion

d) drying the solid dispersion of step (c) until the residual solvents level is well below acceptable limits (as per ICH guideline Q3C (R6),

e) sizing the solid dispersion of step (d) through a sieve of NMT 250 μm mesh size, f) mixing the remaining portion of the total amount of tacrolimus compound and the filler,

g) adding lubricant and blending to form a final blend

h) filling capsules

The following examples illustrate the invention but it should be understood that variations and modifications may be made without departing from the spirit and scope of the invention as defined in the appended claims.

### EXAMPLES

### Example 1

A pharmaceutical formulation as presented in Table 1 below was prepared.

TABLE 1
Pharmaceutical formulation of example 1

| Formulation 1 | % w/w |
|---|---|
| Internal phase | |
| Tacrolimus monohydrate | 0.93 |
| (equivalent to tacrolimus) | (0.91) |
| Ethylcellulose | 0.43 |
| HPMC 2910 | 0.43 |
| Lactose monohydrate | 7.14 |
| External phase | |
| Lactose monohydrate | 90.07 |
| Mg stearate | 1.00 |
| Total blend | 100.00 |

The process of formulation 1 above comprises the following steps:

a) dissolving the total amount of tacrolimus in pure ethanol (anhydrous ethanol) to form a macrolide solution,

b) dissolving the total amount of ethylcellulose in the tacrolimus solution of step (a) and stirring till complete homogenization

c) pre-mixing the hydroxypropylmethyl cellulose with lactose monohydrate,

d) mixing the tacrolimus solution of step (b) with the premixed blend of solid materials of step (c) to form a solid dispersion (paste)

e) drying the solid dispersion of step (d) until the residual solvents level is well below acceptable limits (as per ICH guideline Q3C (R6)),

f) sizing the solid dispersion of step (e) through a sieve of NMT 250 μm mesh size,

g) mixing the remaining portion of the total amount of lactose monohydrate with solid dispersion of step (f),

h) adding magnesium stearate and blending to form a final blend

The in-vitro dissolution profile of Formulation 1 was recorded in Buffer pH 1.2, 100 RPM, 900 mL, USP II with 0.005% hydroxypropylcellulose in comparison to reference product. The dissolution results, showed that the prolonged release profile of tacrolimus for the Formulation 1 shall be optimized to address the burst effect along the early time intervals without suppressing the drug release at later dissolution intervals.

Also, the manufacturing process of formulation 1 shall be optimized since the paste (intermediate product) preparation along the manufacturing process step (d) isn't easily handled and the resulting dispersion cannot be easily sieved after drying step.

### Example 2

The optimization process for the manufacturing process and the formulation variables led the inventors is developing and studying a number of formulations. Formulations 2 to 7 are shown in table 2 below.

TABLE 2
Pharmaceutical formulation 2-7 comprising Tacrolimus

| | Formulation | Formulation | Formulation | Formulation | Formulation | Formulation |
|---|---|---|---|---|---|---|
| | 2 | 3 | 4 | 5 | 6 | 7 |
| | Amount | Amount | Amount | Amount | Amount | Amount |
| Ingredients | (% w/w) | (% w/w) | (% w/w) | (% w/w) | (% w/w) | (% w/w) |
| Tacrolimus | 0,930 | 0,744 | 0,465 | 0,372 | 0,186 | N/A |
| monohydrate | (100% of | (80% of | (50% of | (40% of | (20% of | |
| | stated | stated | stated | stated | stated | |
| | amount) | amount) | amount) | amount) | amount) | |
| Ethylcellulose | 3,000 | 3,000 | 3,000 | 3,000 | 3,000 | 3,000 |
| std 10 | | | | | | |
| HPMC E4M | 10,000 | 10,000 | 10,000 | 10,000 | 10,000 | 10,000 |
| Lactose | 42,535 | 42,535 | 42,535 | 42,535 | 42,535 | 42,535 |
| monohydrate | | | | | | |
| Tacrolimus | N/A | 0,186 | 0,465 | 0,558 | 0,744 | 0,930 |
| monohydrate | | (20% of | (50% of | (60% of | (80% of | (100% of |
| | | stated | stated | stated | stated | stated |
| | | amount) | amount) | amount) | amount) | amount) |
| Lactose | 42,535 | 42,535 | 42,535 | 42,535 | 42,535 | 42,535 |
| monohydrate | | | | | | |
| Magnesium | 1,000 | 1,000 | 1,000 | 1,000 | 1,000 | 1,000 |
| stearate | | | | | | |
| Total weight | 100,000 | 100,000 | 100,000 | 100,000 | 100,000 | 100,000 |
| of capsule | | | | | | |
| content | | | | | | |

The manufacturing process applied along the preparation of aforementioned pharmaceutical preparations 3-6 is the following:

a) dissolving from about 20% to about 80% of the total amount of Tacrolimus compound to be used in the formulation in an organic solvent (anhydrous ethanol) to form a macrolide solution,

b) pre-mixing the hydroxypropylmethyl cellulose and ethyl cellulose with 50% of stated amount of lactose monohydrate,

c) mixing the macrolide solution of step (a) with the premixed blend of solid materials of step (b) to form a solid dispersion.

d) drying the solid dispersion of step (c) until the residual solvents level is well below acceptable limits (as per ICH guideline Q3C (R6)),

e) sizing the solid dispersion of step (d) through a sieve of NMT 250 μm mesh size

f) mixing the remaining portion of the total amount of macrolide compound and lactose monohydrate with the solid dispersion of step (f),

g) adding magnesium stearate and blending to form a final blend

For Trial 2 and Trial 7 all the amount of Tacrolimus was added either at the external phase or the internal phase respectively, following the same manufacturing process.

TABLE 3
Dissolution results for Formulations 2-7 and reference product 5 mg prolonged-
release hard capsules.

| | Drug dissolved (%) | | | | | | |
|---|---|---|---|---|---|---|---|
| | Formulation | Formulation | Formulation | Formulation | Formulation | Formulation | |
| Time | 2 | 3 | 4 | 5 | 6 | 7 | Reference |
| (hr) | 5 mg caps | 5 mg caps | 5 mg caps | 5 mg caps | 5 mg caps | 5 mg caps | product |
| 1 | 41.8 | 37.6 | 32.1 | 28.7 | 23.6 | 17.8 | 25.5 |
| 2 | 55.7 | 43.8 | 34.3 | 30.7 | 24.5 | 20.8 | 28.6 |
| 3 | 62.3 | 45.8 | 36.8 | 34.1 | 27.1 | 24.1 | 32.2 |
| 4 | 65.2 | 47.4 | 38.5 | 36.1 | 28.8 | 26.3 | 34.8 |
| 5 | 68.4 | 52.8 | 41.8 | 37.9 | 31.6 | 27.8 | 38.6 |
| 6,5 | 71.4 | 55.8 | 43.8 | 39.6 | 35.8 | 28.8 | 40.7 |
| 8,5 | 73.9 | 59.5 | 45.9 | 41.7 | 38.4 | 29.5 | 42.5 |
| 12 | 75.6 | 63.5 | 48.2 | 44.2 | 40.1 | 30.4 | 45.8 |
| 16 | 76.8 | 65.8 | 50.6 | 46.4 | 41.5 | 31.0 | 47.9 |
| 24 | 78.4 | 68.2 | 52.4 | 47.5 | 43.2 | 31.5 | 48.6 |

The in-vitro dissolution profile of Formulations 2-7 was recorded in Buffer pH 1.2, 100 RPM, 900 mL, USP II with 0.005% hydroxypropylcellulose in comparison to reference product and the results are shown in table 3 above.

The dissolution results clearly show that the higher API that is non-dissolved within the solid dispersion, the lower the dissolution rate. Taking into account the poor aqueous solubility of Tacrolimus, the current dissolution method can discriminate the different formulation trials suggesting that the formulations can be further investigated.

Since the scope of current pharmaceutical preparations was to investigate the impact of Tacrolimus crystallinity on bioavailability metric under in-vivo conditions, a bioequivalence study was conducted on healthy fasted subjects to determine the bioavailability of the test product (Formulation 5) versus reference product. The results obtained were well within the acceptable range for a bioequivalent product. Moreover, according to the dissolution results (Table 3) reference product exhibits similar in-vitro dissolution profile to Formulation 4-6. Therefore, it is reasonably expected that the in-vivo behavior will be similar for all three formulations 4 to 6.

A stability study was also conducted. Under the scope of the evaluation of the related substances profile of finished dosage form, Tacrolimus prolonged-release capsules, of formulation 5, were loaded into stability chambers and monitored with a fit-for-purpose HPLC analytical method. Stability data (related substances) upon storage at zero time & 6 months under long term (25° C.±2° C./60%±5% RH) and accelerated storage conditions (40° C.±2° C./75%±5% RH). The stability results showed that the formulation is stable since no significant shift of dissolution rate was recorded even in accelerated storage conditions for 6 months. Also, the related substances after 6 months of stability at Long-term & Accelerated storage conditions are well within the acceptance criteria.

1. An oral prolonged release pharmaceutical composition comprising a solid dispersion of Tacrolimus or a pharmaceutically acceptable salt thereof as the active ingredient, wherein the amount of Tacrolimus or a pharmaceutically acceptable salt in the solid dispersion is from about 20% to not more than 50% by weight of the total pharmaceutically effective amount of the active ingredient.

2. The pharmaceutical composition of claim 1, wherein the remaining amount of Tacrolimus or a pharmaceutically acceptable salt is not dissolved or dispersed.

3. The pharmaceutical composition of claim 1, wherein the solid dispersion comprises a hydrophilic and a hydrophobic polymer.

4. The pharmaceutical composition of claim 3, wherein the hydrophilic polymer is hydroxypropylmethyl cellulose and a hydrophobic polymer is ethyl cellulose.

5. The pharmaceutical composition of claim 3, wherein the ratio of the total amount of tacrolimus or pharmaceutically acceptable salt thereof, to the combined amount of the hydrophilic and the hydrophobic polymer is from 1:1 to 1:20.

6. The pharmaceutical composition of claim 1, wherein the composition further comprises a filler and a lubricant.

7. The pharmaceutical composition of claim 6, wherein the filler is lactose.

8. The pharmaceutical composition of claim 6, wherein the lubricant is magnesium stearate.

9. The pharmaceutical composition of claim 1, wherein the formulation is in the form of granules in particulate form, filled in a capsule, with a mean diameter of not more than 250 μm.

10. A process for preparing a pharmaceutical composition according to claim 1, comprising the following steps:
a) dissolving from about 20% to not more than 50% by weight of the total amount of Tacrolimus or a pharmaceutically acceptable salt thereof in an organic solvent to form a Tacrolimus solution,
b) pre-mixing the hydrophilic polymer and the hydrophobic polymer with at least 50% of the filler,
c) mixing Tacrolimus solution of step (a) with the premixed blend of solid materials of step (b) to form a solid dispersion,
d) drying the solid dispersion of step (c) until the residual solvents level is well below acceptable limits (as per ICH guideline Q3C (R6)),
e) sizing the solid dispersion of step (d) through a sieve of NMT 250 μm mesh size,
f) mixing the remaining portion of the total amount of Tacrolimus with the remaining portion of the total amount of the filler,
g) adding lubricant and blending to form a final blend h) filling capsules.

11. The process according to claim 10, wherein the hydrophilic polymer is hydroxypropylmethylcellulose & the hydrophobic polymer is ethyl cellulose.

12. The process according to claim 10, wherein the filler is lactose monohydrate & the lubricant is magnesium stearate.
